(19) United States
(12) Patent Application Publication
(10) Pub. No.: US 2002/0058861 A1
(43) Pub. Date: May 16, 2002

(54) Medical monitoring system

(76) Inventor: Guy A. Drew, San Antonio, TX (US)

Correspondence Address: Llyod Whitworth, Corporate Initiatives Coordinator, Professional Performance Development Group, Inc., Suite A-125, 4241 Woodcock Dr., San Antonio, TX, 78228, US

(21) Appl. No.: 09/809,463

(22) Filed: Mar. 15, 2001

Related U.S. Application Data
(60) Provisional application No. 60/189,967, filed on Mar. 17, 2000.

(51) Int. Cl. A61B 5/00

(52) U.S. Cl. 600/300; 128/903

(57) ABSTRACT

The medical monitoring system provides for the gathering of signals from a variety of medical monitoring equipment that is attached to a patient either human or animal by a medical monitor. The medical monitor passes the gathered signals to a signal distribution system for conditioning, filtering, and multiplexing/switching. The signal distribution system then passes the signals to a computer for analysis, storing, and monitoring. The computer may be located at a central location or hub and be receiving information and signals from multiple medical monitors over a network that may be wireless and/or wired. The computer may instead be connected to just one medical monitor. Depending upon the exact implementation, the exact location of these three components will be dictated.

I hereby claim the benefit under 35 U.S.C. 119(e) of the United States provisional patent application Ser. No. 60/189,967, filing date, Mar. 17, 2000. This nonprovisional patent application is for the same utility as previously declared in provisional patent application Ser. No. 60/189,967.

### FIELD OF THE INVENTION

The field of the invention includes any medical monitoring situation of patients, animals, and/or clinical/laboratory/research.

1. Background of the Invention

Previous systems required different components to perform filtering, anti-aliasing, signal conditioning, and/or amplification. Researchers are required to use different systems when the monitoring equipment is provided by different companies, because each company uses a proprietary system that prevents them from talking with each other.

2. Summary of the Invention

The medical monitoring system provides for the gathering of signals from a variety of medical monitoring equipment that is attached to a patient either human or animal by a medical monitor. The medical monitor passes the gathered signals to a signal distribution system for conditioning, filtering, and multiplexing/switching. The signal distribution system then passes the signals to a computer for analysis, storing, and monitoring. The computer may be located at a central location or hub and be receiving information and signals from multiple medical monitors over a network that may be wireless and/or wired. The computer may instead be connected to just one medical monitor. Depending upon the exact implementation, the exact location of these three components will be dictated.

This system may be utilized in a research setting to take medical readings from a subject. The system may include a calibrator to calibrate the signals obtained from the signals obtained from the medical readings to adjust the signal intake so that useful data may be taken and stored. The calibration feature may also be used to troubleshoot the equipment taking the medical readings.

This system is able to record physiological signals on patients in either an operating room and/or a recovery room. The system may interface and incorporate equipment manufactured by, for example, Hewlett Packard, Ohmeda, or Baxter.

An objective of this invention is to provide real-time access to analysis of medical data to medical professionals along with the basic medical information.

### BRIEF DESCRIPTION OF THE DRAWINGS

FIG. 1 illustrates a front view of the preferred medical monitor arrangement.

FIG. 2 depicts a side view of the preferred medical monitor arrangement.

FIG. 3 illustrates a top view of the chassis of the preferred medical monitor arrangement.

FIGS. 4(a)-(c) depict the I/O interface of the preferred medical monitor arrangement.

FIG. 5 illustrates a side view of the preferred medical monitor arrangement attached to medical monitoring equipment a wire harness.

FIG. 6 depicts a front view of the data acquisition system including the signal distribution system of the preferred embodiment of the invention.

FIG. 7 illustrates a front view of the data acquisition system including the signal distribution system of the preferred embodiment of the invention.

FIG. 8 depicts a side view of the data acquisition system of the preferred embodiment of the invention.

FIG. 9 illustrates the I/O interface of the preferred data acquisition system.

FIG. 10 depicts a second I/O interface of the preferred data acquisition system.

FIG. 11 illustrates an interior view of the data acquisition system chassis with the door removed.

FIG. 12 depicts the interior view of the data acquisition system with the door open.

FIG. 13 illustrates the top view of the data acquisition system of the preferred embodiment of the invention.

FIGS. 14(a)-(b) depict the monitor output of the data acquisition system.

FIGS. 15(a)-(b) illustrate the front and rear, respectively, of the enlarged displays.

FIG. 16 depicts a functional interconnection between components of the data acquisition system of the preferred embodiment of the invention.

FIG. 17 illustrates a functional scheme of components of the data acquisition system of the preferred embodiment of the invention.

FIGS. 18(a)-(b) depict the connections of the signal distribution system of the preferred embodiment of the invention.

FIG. 19 illustrates a perspective view of the signal distribution system combined with the meter display panel and calibration meter.

FIG. 20 depicts a functional scheme of the signal distribution system.

FIG. 21 illustrates a circuit diagram of the signal distribution system cards.

FIG. 22 depicts a component diagram for the card illustrated in FIG. 21.

FIG. 23 illustrates a component diagram of the back surface of the switching matrix of the signal distribution system.

FIG. 24 depicts an alternative embodiment as a block diagram.

### DETAILED DESCRIPTION OF THE DRAWINGS

Like reference numerals in the figures represent and refer to the same element or function. In communication in this document includes directly connected, connected via other elements or components (i.e., indirectly), and wirelessly.

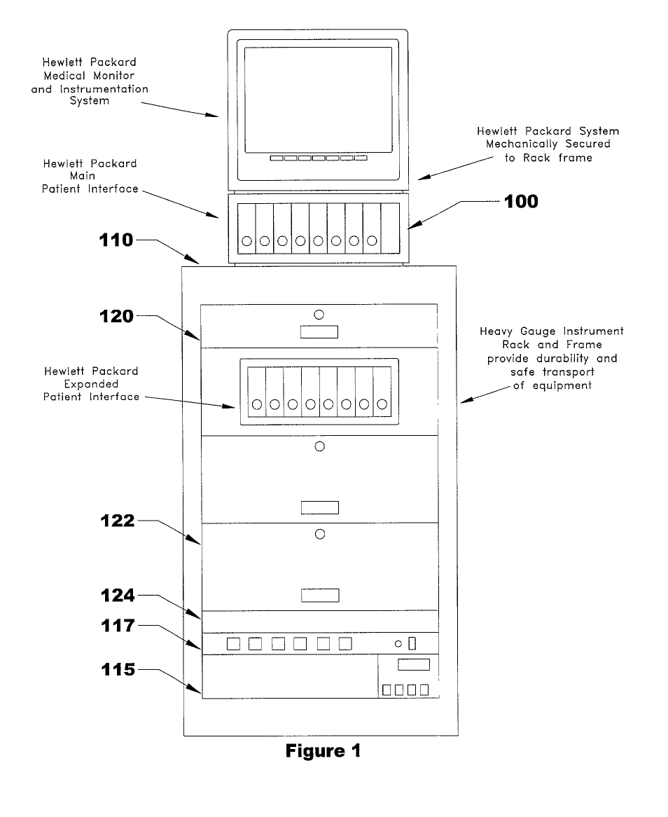

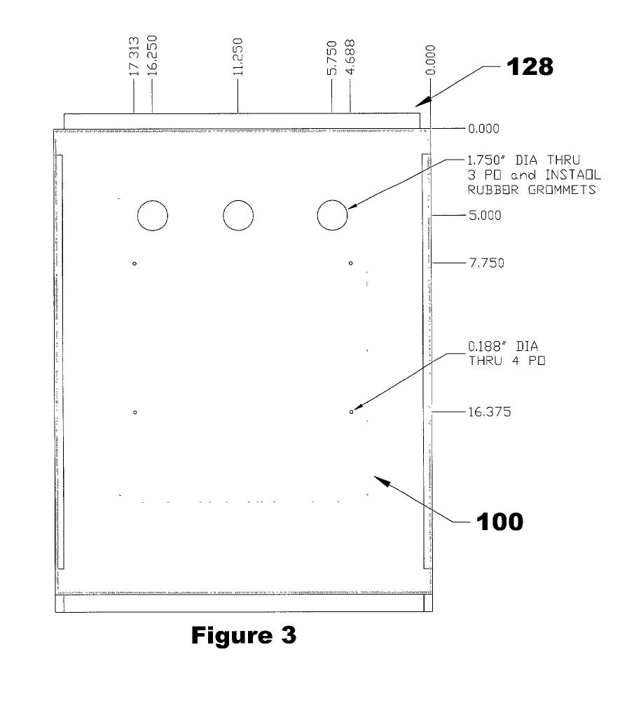

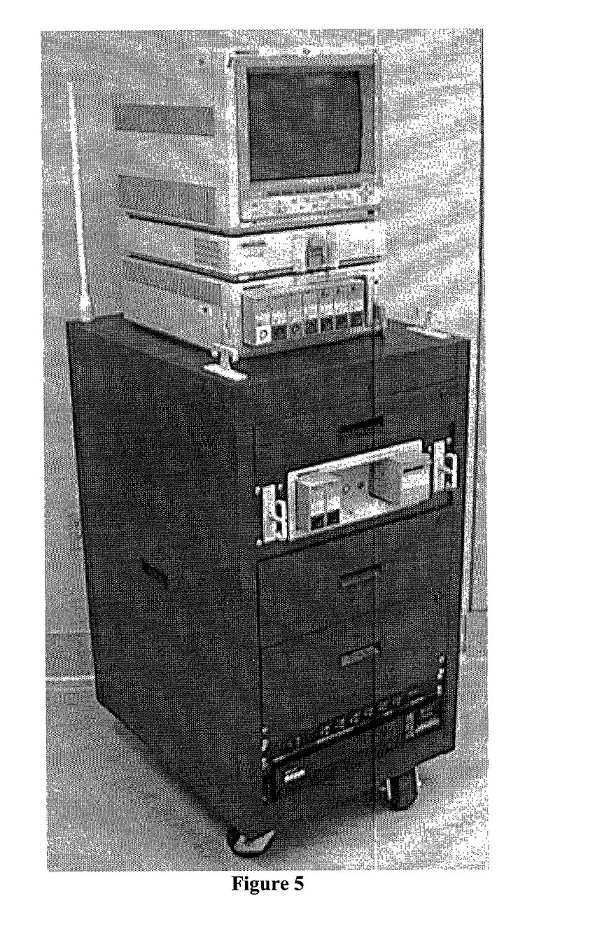

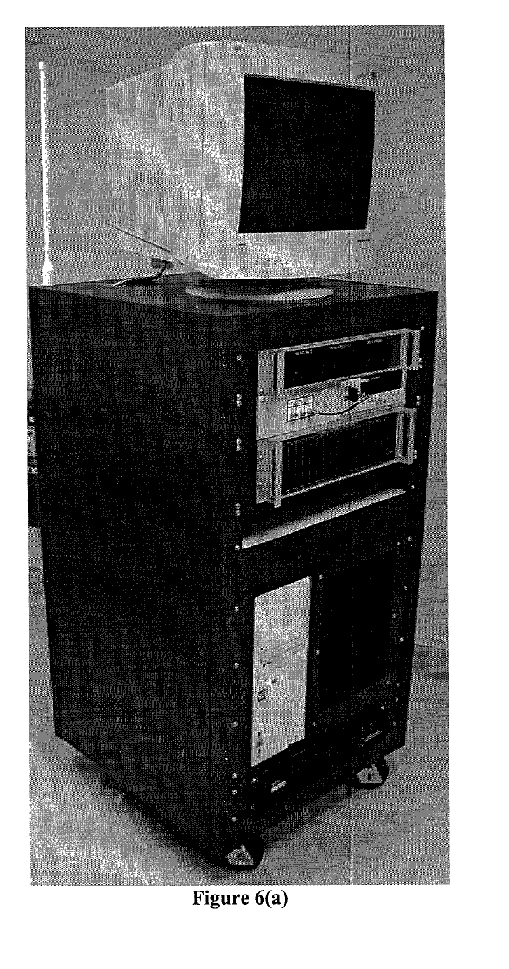

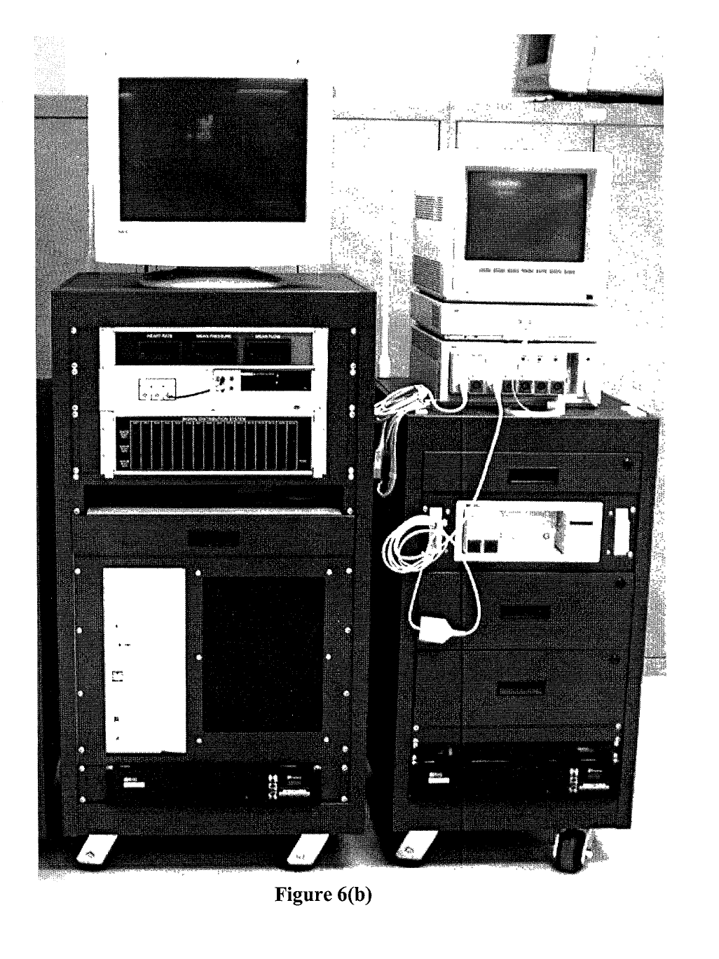

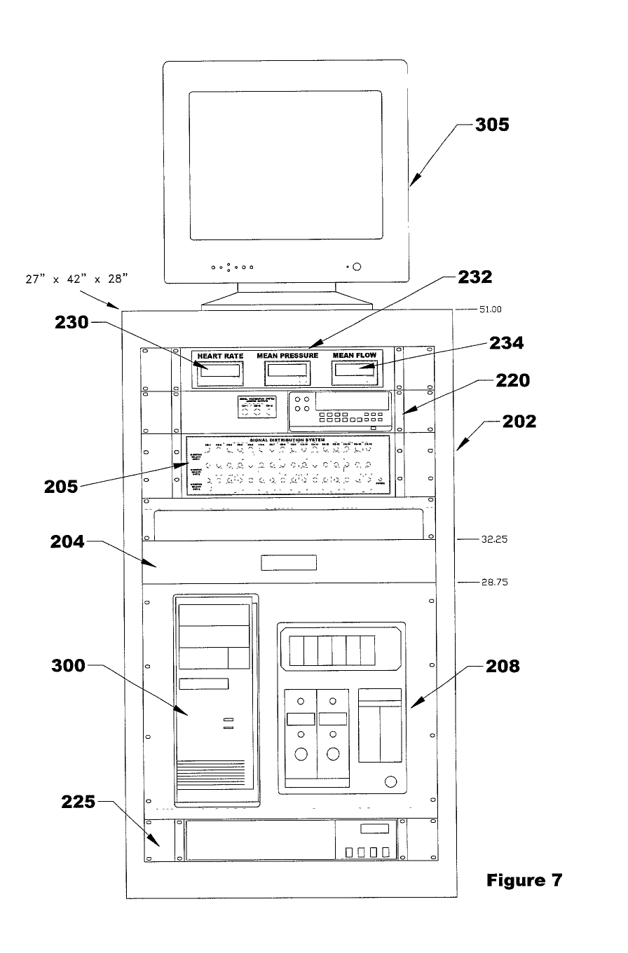

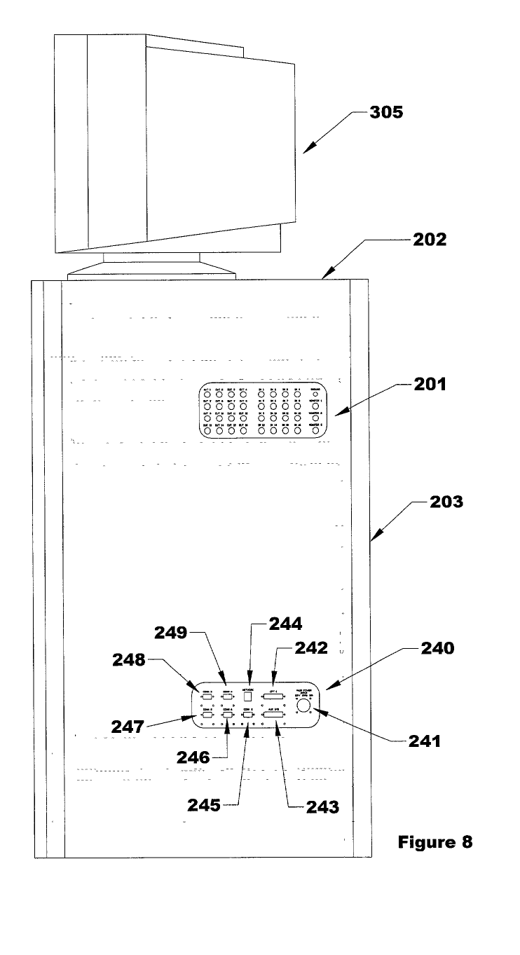

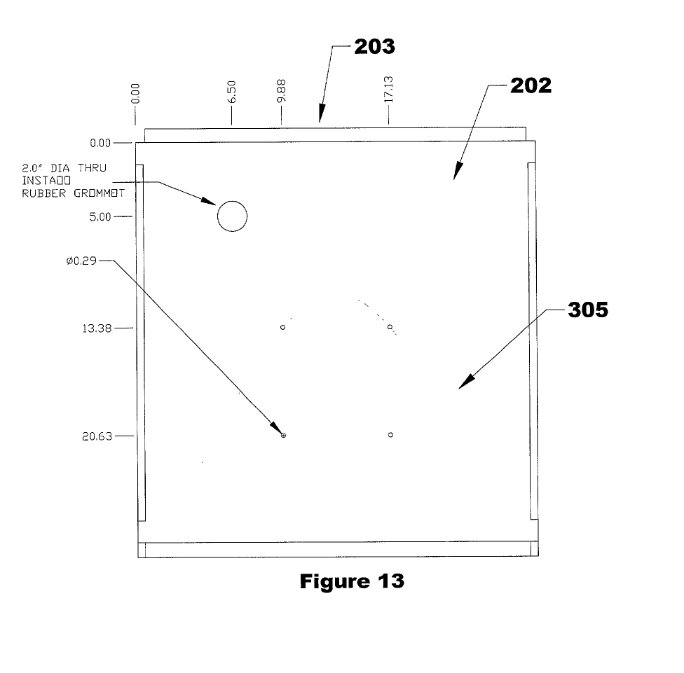

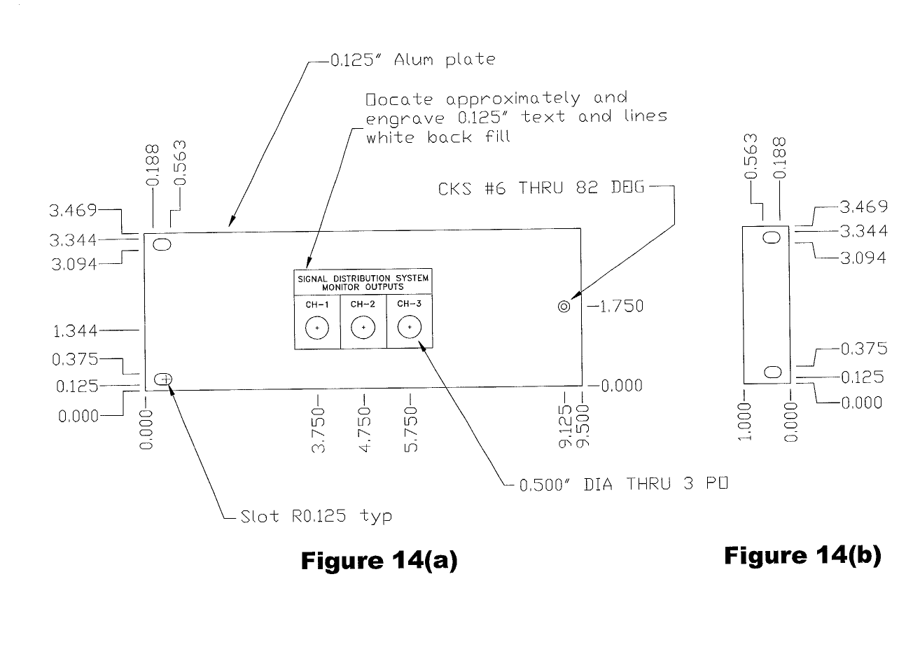

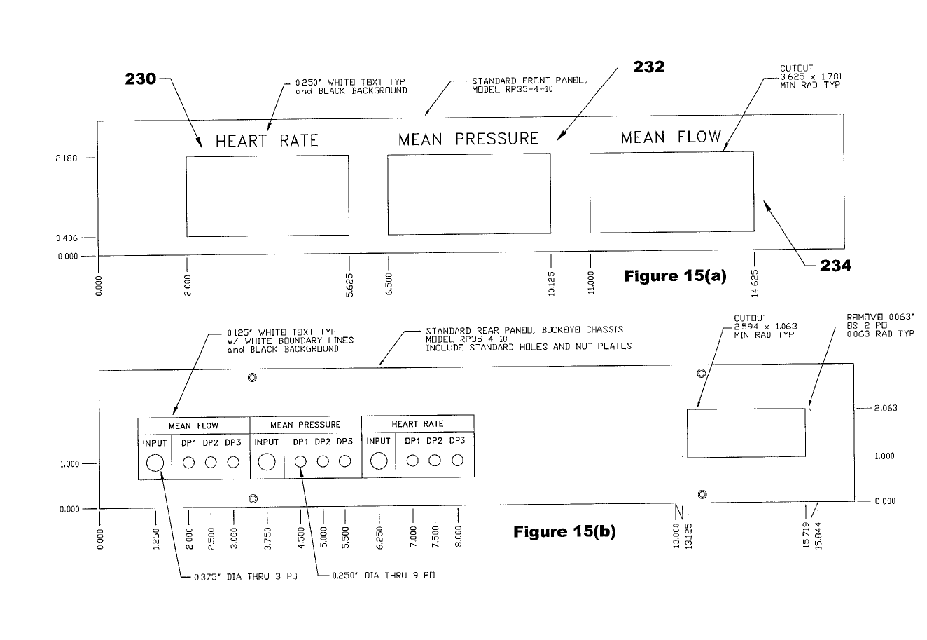

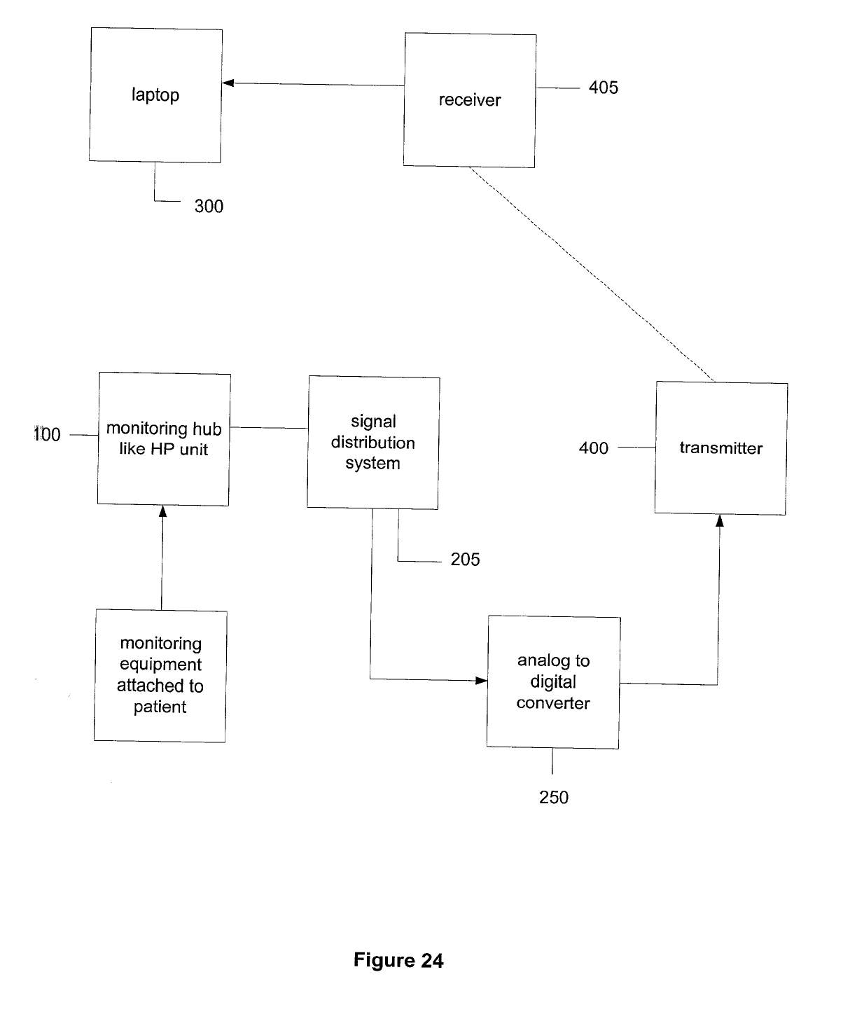

The medical monitoring system preferably includes a medical monitor 100 and a data acquisition system 200. The data acquisition system 200 preferably includes a signal distribution system 210 and a computer 300. FIGS. 1-24 illustrate different components of the medical monitoring system, more particularly FIGS. 1-5 illustrate the medical monitor 100 and FIGS. 6-24 illustrate the data acquisition system 200.

In the preferred embodiment, the medical monitor 100 preferably connects to a wire harness 190 via a first I/O interface 105. The wire harness 190 then preferably connects to the data acquisition system 200 through a first I/O interface 201. The medical monitor 100 preferably is mounted on a chassis 110. FIG. 1, for example, illustrates the medical monitor 100 as a Hewlett Packard Main Patient Interface and a Hewlett Packard Expanded Patient Interface. One of ordinary skill in the art will realize based on this disclosure that other patient monitoring equipment may be used instead of the illustrated Hewlett Packard Interface.

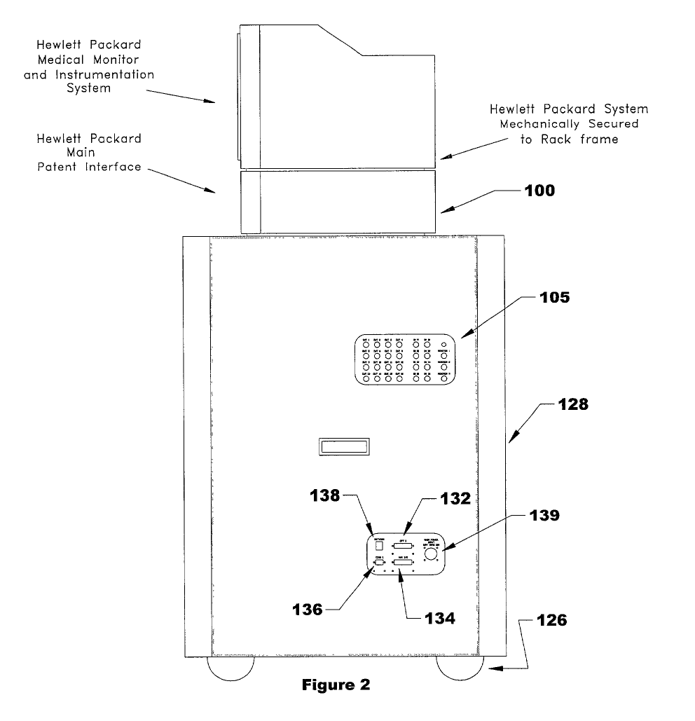

The chassis 110 preferably includes a power supply 115. Preferably, the power supply 115 is a uninterruptible power system that filters wall power and provides battery backup power, if needed, and provides additional outlets 117 for connecting other equipment that may be needed. The chassis 110 preferably also will include a plurality of drawers 120, 122, 124 for storage and wheels 126, illustrated in FIG. 2, on the bottom surface to move the chassis 110. The chassis 110 preferably also will have a door 128 illustrated for example as being a rear door to protect equipment housed within the chassis 110.

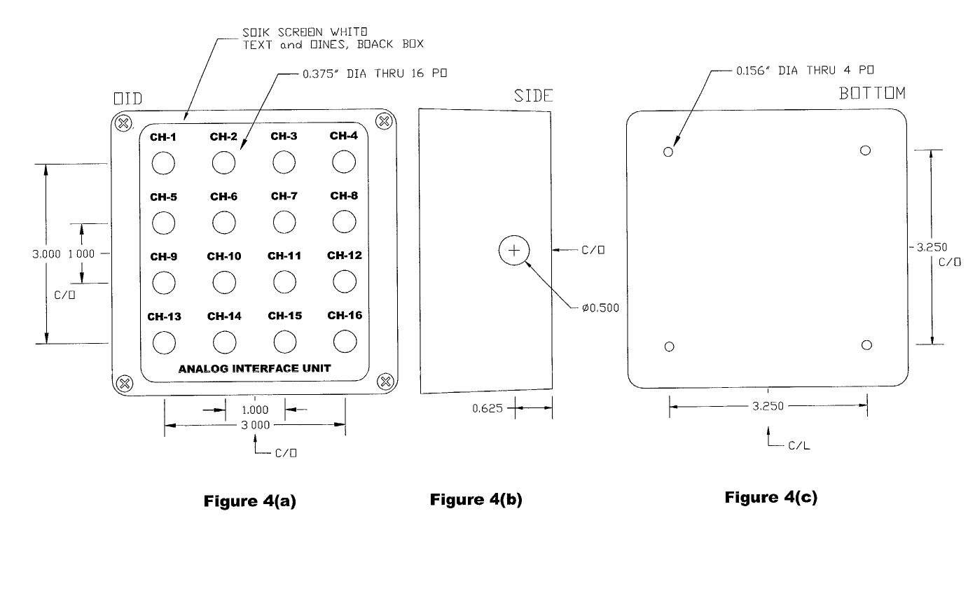

The first I/O interface 105 preferably provides a signal interface to connect the wire harness 190 for transmitting signals to the data acquisition system 200. FIGS. 4(a)-(c) illustrate an example of construction of a box for the 16 channels of the first I/O interface 105. A second I/O interface 130 preferably also is provided for communication with other equipment through communication ports 132, 134, 136, 138 similar to those that are found on computers and a main power interface plug 139.

FIG. 12-23 illustrate a preferred data acquisition system 200. The embodiment illustrated in FIG. 6 is for a research and/or troubleshooting environment, primarily because the system includes a calibrator 220 such as a Fluke 45 Precision digital meter. Preferably the data acquisition system 200 is housed in a chassis 202 that provides for space for equipment and components useful for monitoring patients and conducting research. However, the data acquisition system 200 may include in addition to the signal distribution system 210 and the computer 300 the following components: large displays 230, 232, 234 and calibrator 220. The chassis 202 preferably includes a power supply 225, which preferably includes EMI/FRI filters and uninterruptible power supply, and a drawer 204. An alternative embodiment adds pressure, flow and ECG equipment 208.

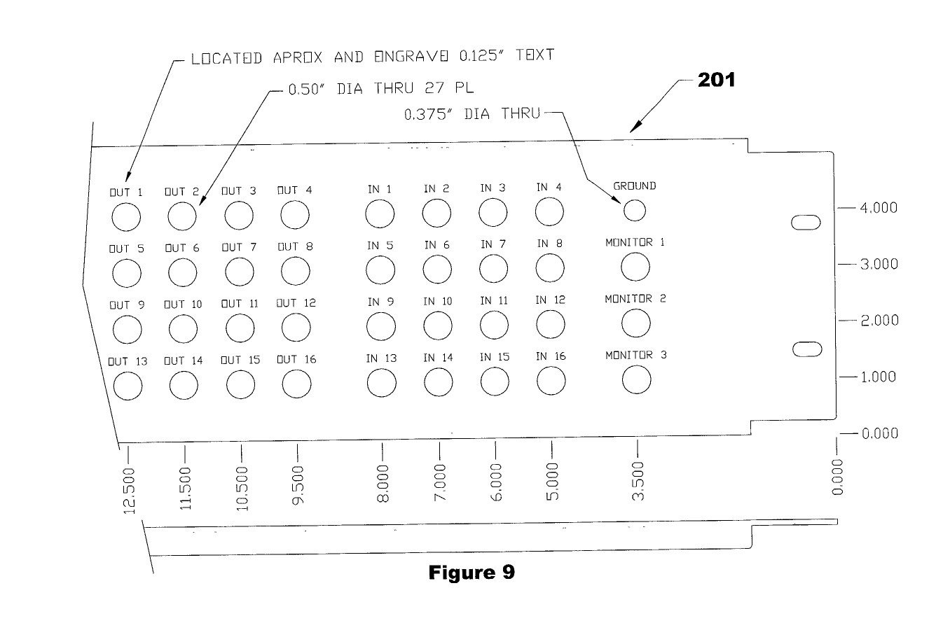

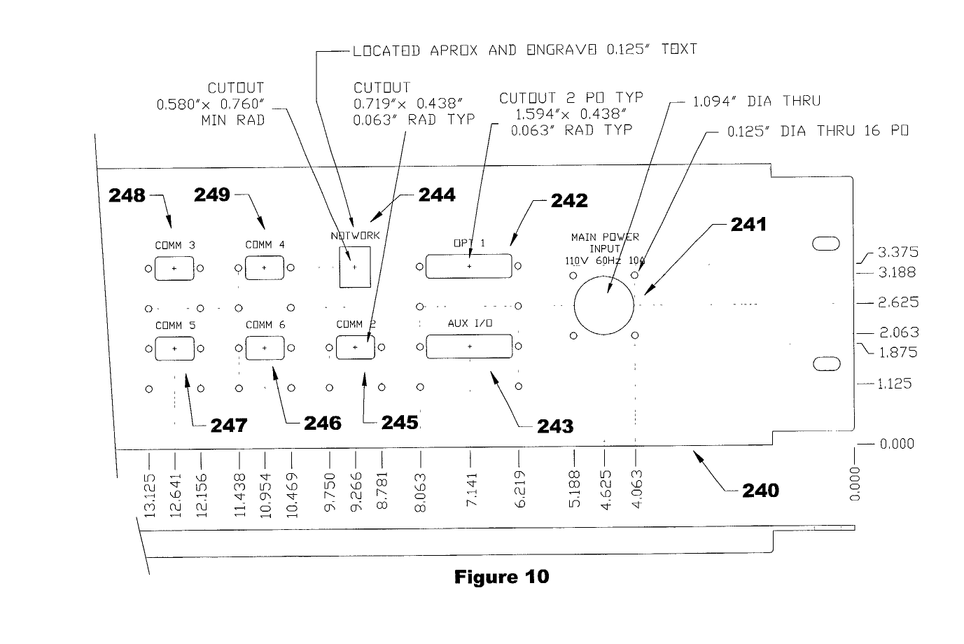

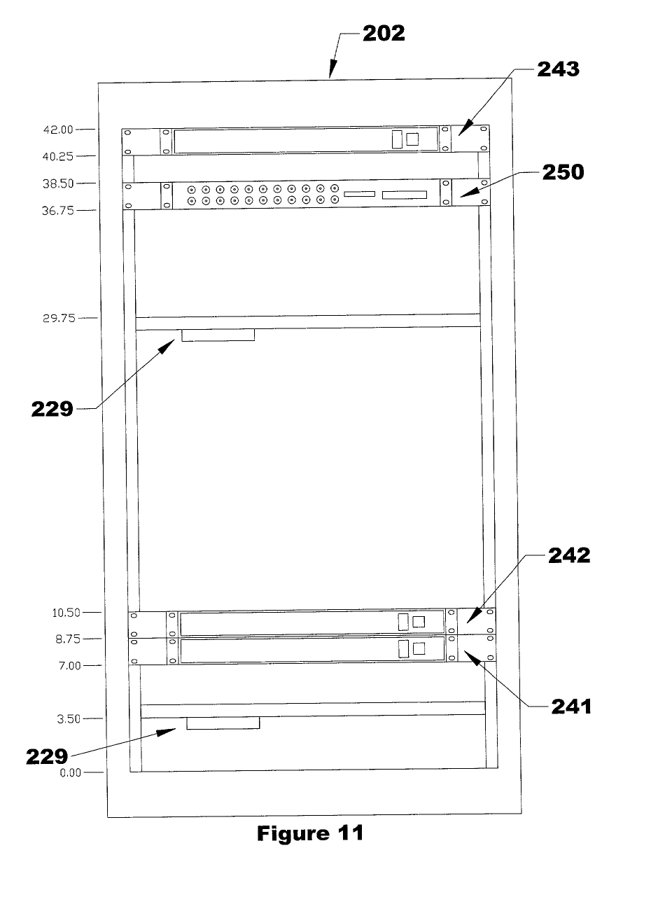

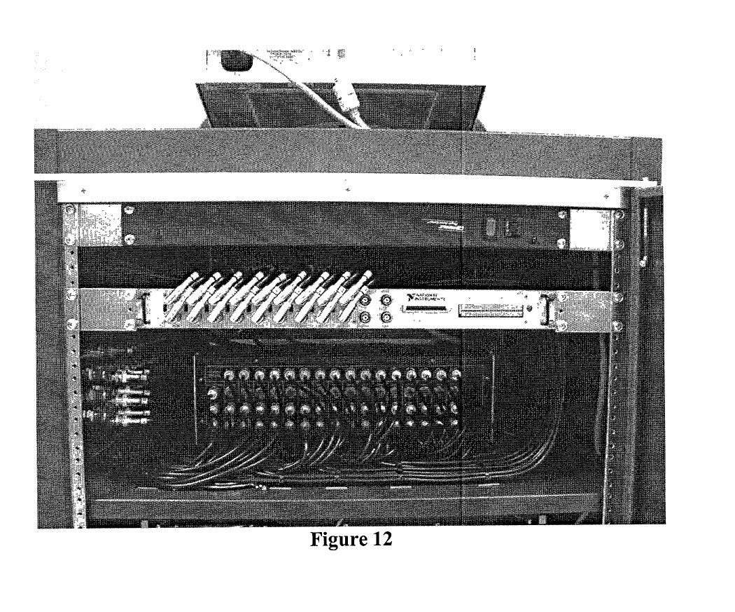

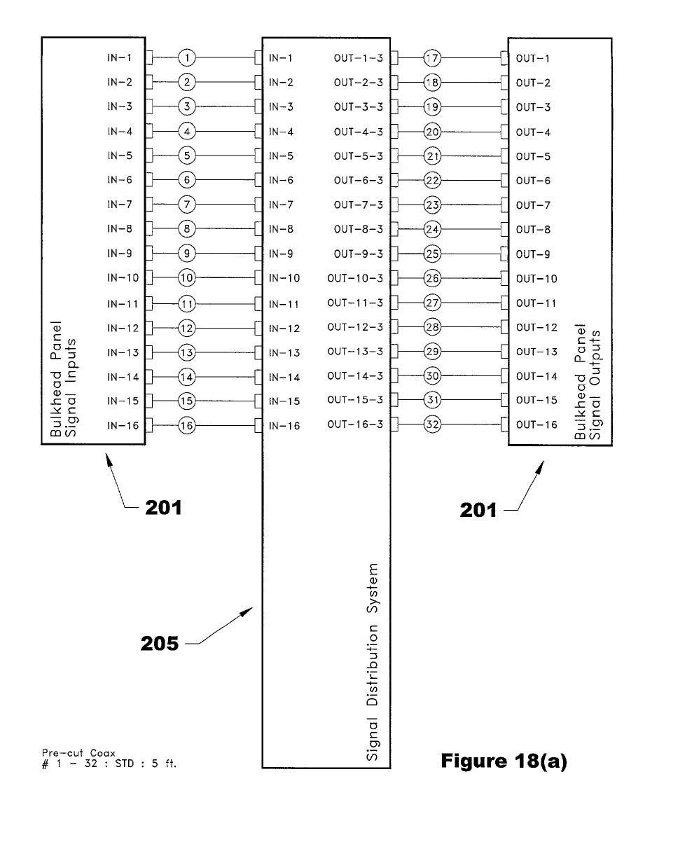

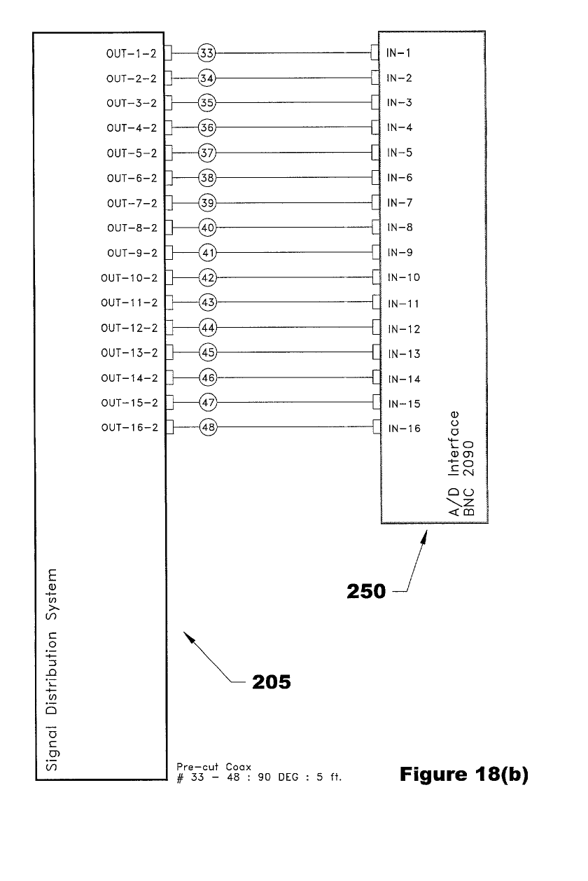

The first I/O interface 201 preferably includes two sets of sixteen connectors to act as an input and the other as the output. Also, preferably the first I/O interface 201 includes a ground, monitor 1, monitor 2, and monitor 3 as illustrated in FIG. 9. A second interface 240 preferably includes a power input 241, a LPT 1 jack 242, and auxiliary slot 243, a network jack 244, and five communication jacks 245, 246, 247, 248, 249 as illustrated, for example, in FIG. 10. The network jack 244 will allow for connection of the data acquisition system 200 to an external computer network. The chassis 202 preferably will also have a door 203 to protect the electrical equipment housed within the chassis 202 such as the signal distribution system 210. Internal to the chassis 202 as illustrated, for example, in FIG. 11 may be power strips 226, 227, 228 and a cooling fan 229. The data acquisition system preferably will include an analog/digital converter 250 such as a National Instruments BNC-2090. The analog/digital converter 250 connects to the signal distribution system 210 as illustrated in FIGS. 12 and 18(b) with sixteen 90 degree coaxial cable 33-48 with a length of five feet.

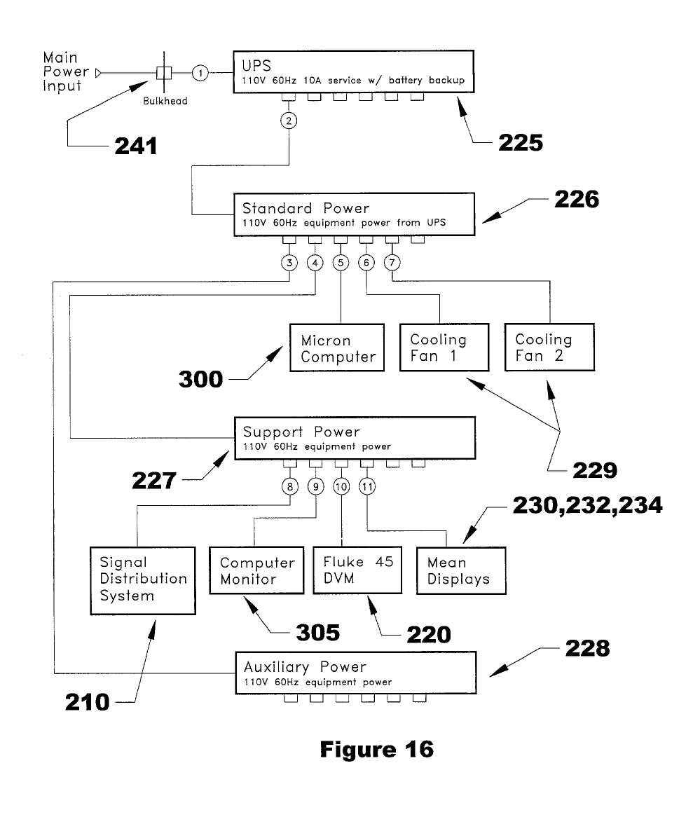

FIG. 16 illustrates the power connections of the data acquisition system 200. The power supply 225 receives power through power input 241. The power supply 225 preferably has the following characteristics: 110 V, 60 Hz, 10 A and battery backup. The optional power strips 226, 227, 228 also preferably provide 110 V with a frequency of 60 Hz. The power supply 225 drives power strip 226, which in turn drives power strips 227, 228, the computer 300, the cooling fan 229. The power strip 227 preferably drives the signal distribution system 210, the computer monitor 305, the calibrator 220, and the displays 230, 232, 234.

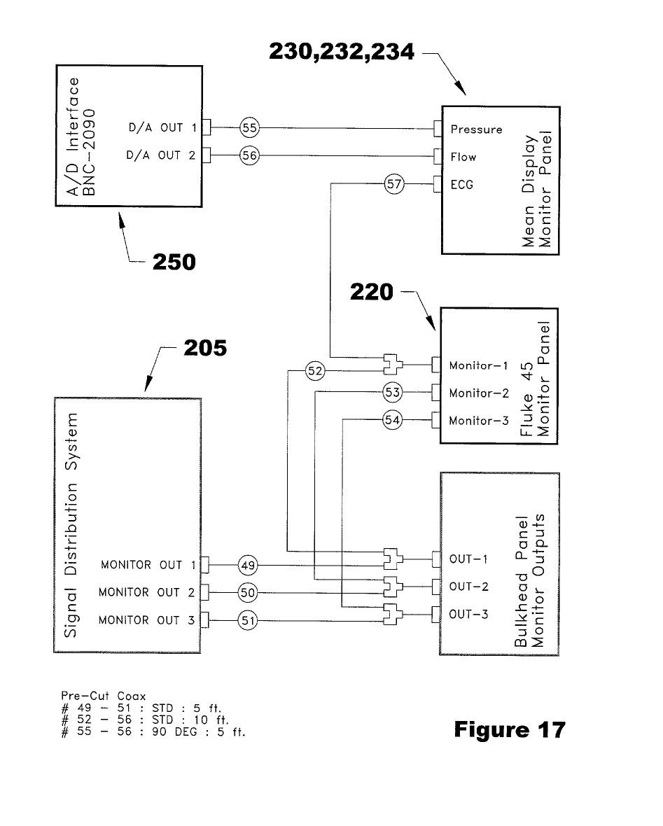

The signal distribution system 205 preferably includes the monitor outputs that connect in series with the bulkhead panel monitor outputs 265, the calibrator 220, the displays 230, 232, 234, and the analog/digital converter 250 as illustrated in FIG. 17. Preferably, the connections are done with coaxial cable. Preferably, cables 49-51 are standard coaxial cable with a length of five feet, cables 52-54, 57 are standard coaxial cable with a length of ten feet, and cables 55-56 are 90 degree coaxial cable with a length of five feet.

The signal distribution system 205 preferably connects with the first I/O interface 201 with thirty-two pieces of standard coaxial cable 1-32 with a length of five feet as illustrated in FIG. 18(a).

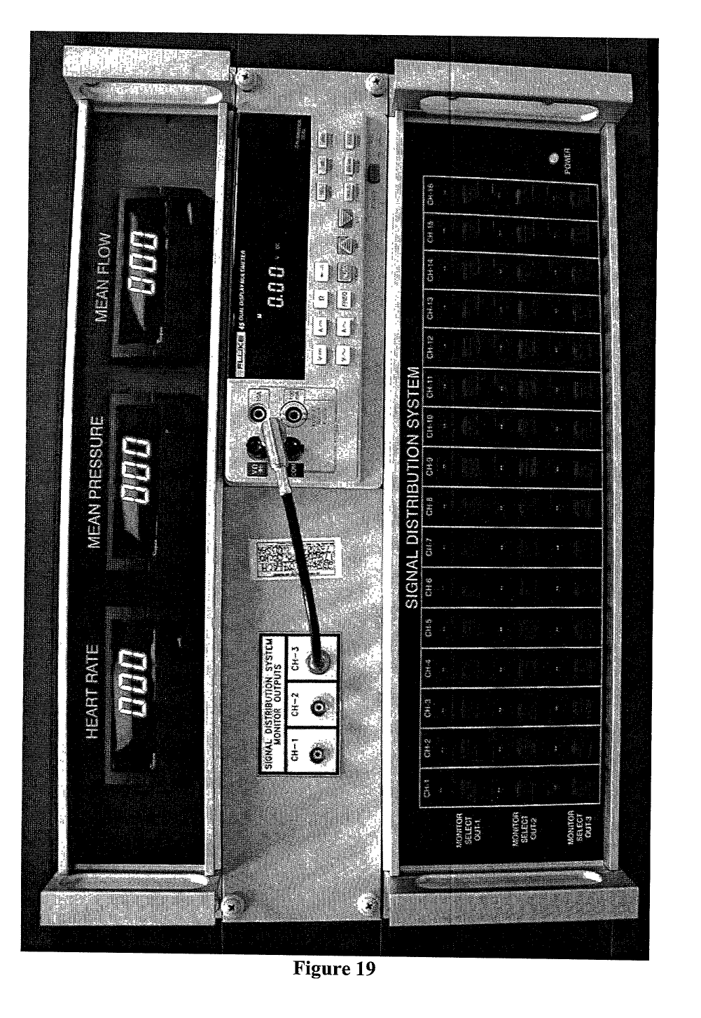

The signal distribution system 205 preferably includes sixteen cards 2052. Each of the cards 2052 preferably performs the following functions: signal-conditioning amplification with selectable differential input, gain, offset and inversion; anti-alias filtering; and cross switch channel multiplexing to monitor selected channels. Each card also preferably provides selectable single ended and differential input mode with the single ended referenced to ground (GND). The cards also preferably provide selectable positive and negative offsets, selectable signal inversion, selectable gain and anti-alias filtering. The gain preferably is selectable through circuit card switches in increments of 2, 3, 4, or 5 times amplitude of the input signal. The cards 2052 preferably are along the rear side 2054 so that the interfaces can be accessible as shown, for example, in FIG. 19.

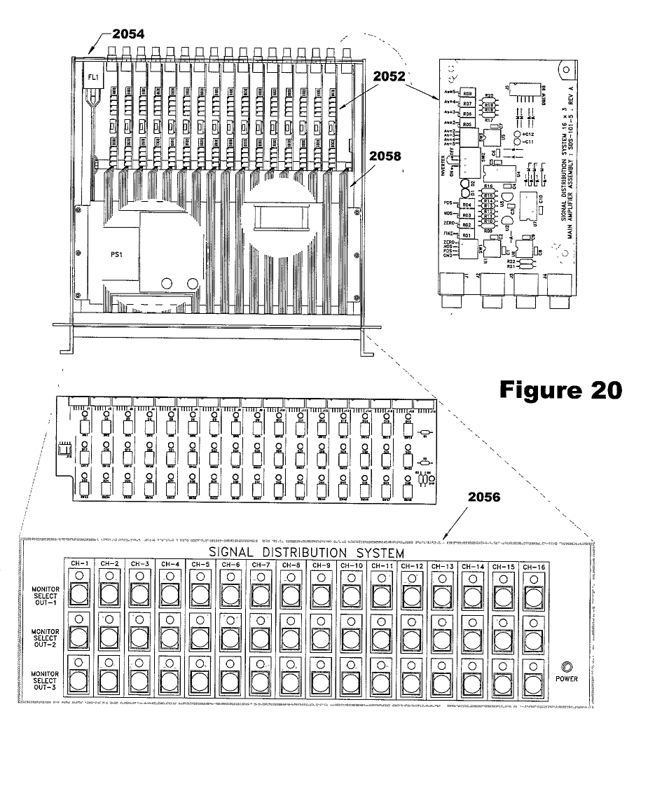

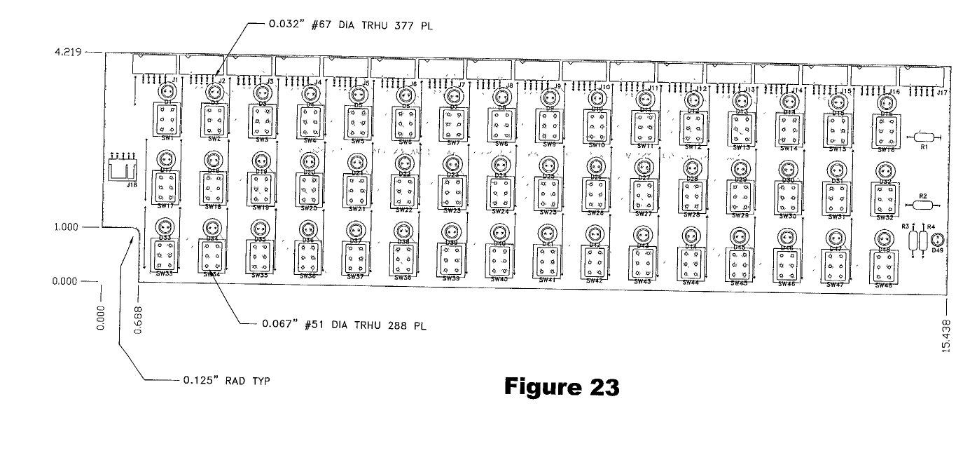

Preferably, the signal distribution system 205 preferably provides three selectable monitor outputs that are controlled by the front panel binary switches. The front panel 2056 preferably includes three rows of monitor select switches with each row having a switch for each channel running through the signal distribution system 205. For example, row number 1 corresponds to monitor out 1, row number 2 corresponds to monitor out 2, and row number 3 corresponds to monitor out 3. Preferably, the front panel switches are high channel priority such that if both channels 5 and 12 in the same row are selected then channel 12 will be displayed on the monitor for that row. A wiring diagram of the rear side of the front panel 2056 is illustrated in FIG. 23. Preferably as illustrated in FIG. 20, there is an array of LEDs that corresponds to the switching matrix such that when a switch is activated the corresponding LED lights. Alternatively, the panel switches may instead operate under low channel priority.

The calibration specifications for the signal distribution system 205 preferably are zero set (0 VDC+/−0.5 mV), positive offset (POS+4 VDC+/−0.5 MV), negative offset (NOS+4 VDC+/−0.5 mV), gain (Av)=1, 2, 3, 4, and 5 (+/−0.1%), inverter (+/−1 mV between inversion), filters (60 Hz with 24 dB/octive), and monitor outputs (+/−1.0 MV of inputted signal). FIGS. 20-23 illustrate cards 2052 in different ways. The legend for the circuit elements is as follows:

| Reference | Description | Part Number | Manufacture |
|---|---|---|---|
| C1 thru C10 | Capacitor, 0.1 uF ceramic | CK05BX104K | Mallory |
| C11, C12 and C13 | Capacitor, 10 uF 25 V tantalum | T352E106K025AS | Kemet |
| D1 and D2 | LED, red T1-3/4 | HLMP-3301 | HP |
| J1, J2, J3 and J4 | Connector, BNC, RA PC mount | 227161-3 | Amp |
| J1, J2, J3 and J4 | Nut, BNC Connector | 1-329631-2 | Amp |
| J1, J2, J3 and J4 | Washer, BNC connector | 1-329632-2 | Amp |
| J5 | Connector header, 10 pin RA | IDH-10LP-SR3-TR | Robinson Nugent |
| Jumper-A2 | Jumper wire, 0.2″ 22 solid | 923345-02 | 3M |
| Jumper-A4 | Jumper wire, 0.4″ 22 solid | 923345-04 | 3M |
| Jumper-A5 | Jumper wire, 0.5″ 22 solid | 923345-05 | 3M |
| Jumper-M4 | not installed | | |
| Jumper-M5 | not installed | | |
| R1 | Resistor, trimmer RA 200 ohms | 3299X-1-201 | Bourns |
| R2 and R8 | Resistor, trimmer RA 2K ohms | 3299X-1-202 | Bourns |
| R3 thru R7 | Resistor, trimmer RA 5K ohms | 3299X-1-502 | Bourns |
| R9 | Resistor,1% film 5.11K ohms | RN55C-5111F | Dale |
| R10 | Resistor, 1% film 4.99K ohms | RN55C-4991F | Dale |
| R11 and 14 | Resistor, 1% film 1.00K ohms | RN55C-1001F | Dale |
| R12 and R13 | Resistor, 1% film 1.21K ohms | RN55C-1211F | Dale |
| R15 and R16 | Resistor, 1% film 432 ohms | RN55C-4320F | Dale |
| R17 | Resistor, 1% film 47.5K ohms | RN55C-4752F | Dale |
| R18 | Resistor, 1% film 22.1K ohms | RN55C-2212F | Dale |
| R19 | Resistor, 1% film 15.0K ohms | RN55C-1502F | Dale |
| R20 | Resistor, 1% film 12.1K ohms | RN55C-1212F | Dale |
| R21 | Resistor, zero ohm | CD1/4W | SEI |
| R22 | Resistor, 1% film 10.0K ohms | RN55C-1002F | Dale |
| SW1 and SW3 | Switch, 4 position dip | BP04KE | C&K |
| SW2 | Switch, 2 position slide, RA | L202-021-MA04B | C&K |
| U1, U2, U3, U5, U6 | Dip socket, machined 8 pin | ICE-083-S-TG | Robinson Nugent |
| U1, U2, U5 and U6 | IC, Instrumentation amp | AD620AN | Analog Devices |
| U3 | IC, Precision 5 V reference | AD586LN | Analog Devices |
| U4 | IC, Low pass Anti-Alias Filter | D74L4B-60 Hz | Frequency Devices |
| U7 | Dip socket, machined 14 pin | ICE-143-S-TG | Robinson Nugent |
| U7 | IC, Precision driver amp | AD704JN | Analog Devices |
| | Printed Circuit Board | SDS-102-5(B) | Circuit CAD |
| | Mounting clip, steel 6-32 RA | 617 | Keystone |
| | Screw, 6-32 × 0.250 panhead SS | 91772-A145 | Mc Master CARR |
| | Washer, #6 locking star tooth SS | 98449-A007 | Mc Master CARR |

Preferably, ribbons cables 2058 connect the various cards 2052 to the switching matrix of front panel 2056. One of ordinary skill in the art will realize based on the above table that a different mix of circuit components may be designed to perform the same function. Furthermore, other manufacturers' components may be used than those listed above for exemplary purposes.

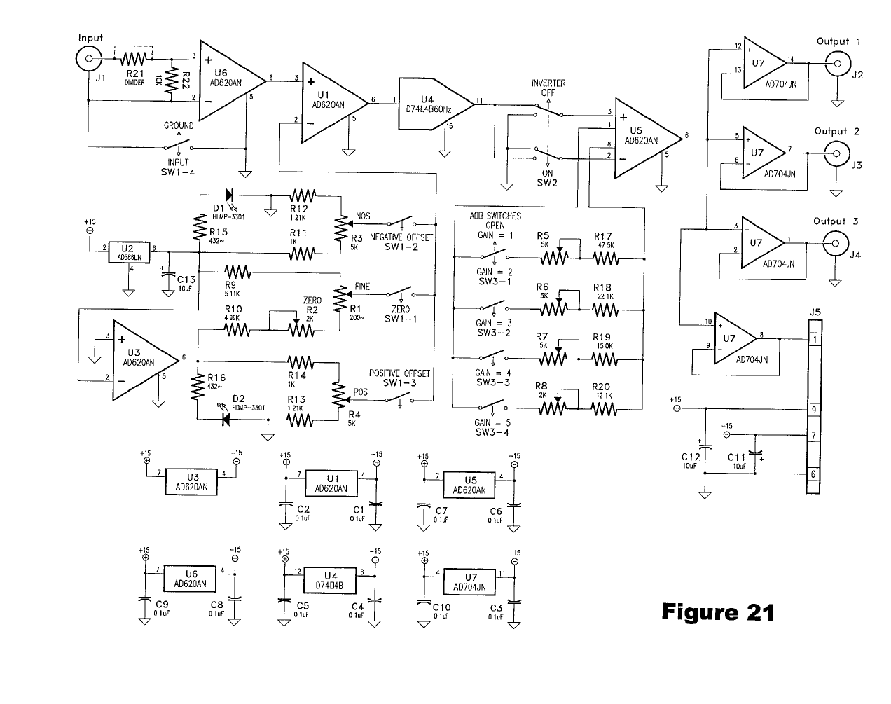

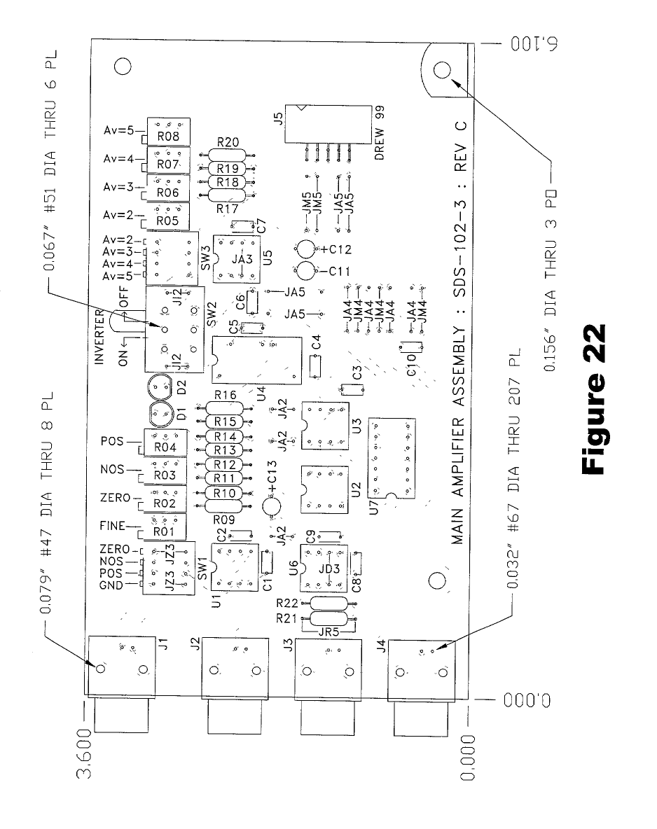

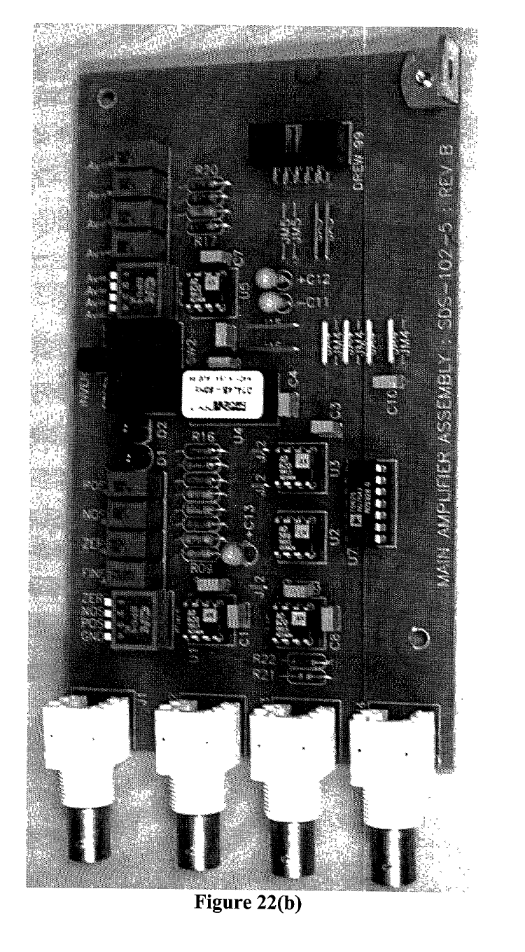

A schematic of the main amplifier driver for each channel consisting of five amplifier stages is shown in FIG. 21. The analog input signal (J1) is initially fed into a differential input amplifier (U6-AD620AN) that can be configured to operate in single-ended or differential mode (Analog Devices, Norwood, Mass.). The second stage consists of positive or negative offset and zero adjust networks that are switch selectable (SW1-1 to SW1-3). These networks also include voltage regulators (U2 and U3) to minimize external noise and influences. For example, the negative offset network includes a resistor combination (R12-R3-R11) that can be configured to provide up to −4 V_{DC} offset. The offset and/or zero networks are fed into a second amplifier (U1). The third stage provides anti-alaising with low pass filters (U4-D74L4B60 Hz) that preferably have a cutoff frequency of 60 Hz with a 24 dB/octave roll off (Frequency Devices, Haverhilll Mass.). Assuming a minimal sampling rate of 200 Hz, which is common for most medical applications, then by the Nyquist Criteria (freq_{sampling}>2×freq_{cutoff}) physiological data will not be Aliased. The user has the option to then invert (SW2) the filtered output, before it is Fed into a third amplification stage that provides user-selectable gain (SW3-1 to SW3-4) from 1× up to 5× in steps of 1×. The output of stage three is then fed through a series of buffer drivers containing precision bipolar amplifiers (U7-AD704JN) that can drive multiple output devices. An illustration of out component level board Layout is shown, for example, in FIGS. 20 and 22.

The computer 300 as illustrated in FIG. 6 is a Millennia Micron computer. The computer 300 collects the date and processes it preferably using software such as LabView. The software provides display of various incoming signals along with real-time calculation of statistics useful to medical professionals and/or researchers.

Preferably, the entire medical monitoring system allows for a pass through rate of equivalent to at least that of a 1 megabyte bus.

An alternative embodiment is a wireless arrangement such that the medical monitor 100 is connected directly to a signal distribution system 205, which then is connected to an analog/digital converter 250. The analog/digital converter 250 connects to a transmitter 400 to send the data wireless to a receiver 405 connected to a computer 300. The telemetry components preferably are any type that allows for transmissions of at least 200 samples per second to form 30 second data sets. An Example is the telemetry equipment available from ViaSat (Carlsbad, Calif.), which equipment is for the purpose of transmitting e-mail not real-time data. This arrangement will be useful for transmitting real-time information from medivac flights to the destination medical facility.

A further alternative embodiment is to form a network of multiple medical monitors 100 that communicate wireless with a central computer 300 such that a medical professional will be able to monitor a group of patients from a central site. This type of arrangement will allow for a medical professional to stay seated on medical transport flight with multiple patients. The central computer in this type of arrangement likely would be a laptop such that the medical professional could scroll through all of the monitoring data of all patients. Preferably, the laptop will have an alarm to be sounded when a patient's medical information degenerates. Another implementation of this type of network is for a medical professional to monitor individuals in the workplace or soldiers on the battlefield to allow for quicker response time to injuries and life threatening medical situations.

An additional embodiment is the placement of a monitoring system onto individual soldiers to monitor their physiological state and transmitting the information via the compact processor/transmitter discussed in the previous paragraph.

Preferably, in either of the alternative embodiments discussed in the previous three paragraphs, the signal distribution system 205 would be stripped down to the card 2052. Even more preferably, the card 2052 is combined with the analog/digital converter 250 and the transmitter 400 such that entire package is compact and easily attached to the medical monitor 100 or other medical monitoring equipment. One of the ordinary skill is in the art will realize based on this disclosure that wireless includes, for example, via satellite, line-of-sight, digital, spread spectrum, PCS, PCMA, DAMA, UHF, VHF, frequency or amplitude modulated, or optically.

Those skilled in the art will realize based on this disclosure that some components may be eliminated from the preferred embodiment and those features illustrated in the attached drawings.

Those skilled in the art will appreciate that various adaptations and modifications of the above-described devices and steps can be configured without departing from the scope and spirit of the their use in the method. Therefore, it is to be understood that, within the scope of the appended claims, the method my be practiced and arranged other than as specifically described herein.

I claim:

1. A system for attaching to medical monitor equipment comprising:
a medical monitor, and
a data acquisition system in communication with said medical monitor.

2. The system according to claim 1, wherein said data acquisition system including a signal distribution system and a microprocessor in communication with said signal distribution system.

3. A system for attaching to medical monitor equipment comprising:
a medical monitor,
a processing system in communication with said monitor,
a converter connected to said processing system, and
an analysis processor in communication with said converter.

4. The system according to claim 3, further comprising:
a first antenna connected to said converter, and
a second antenna connected to said analysis processor and in communication with said first antenna.

5. The system according to claim 4, wherein said first antenna communicates with said second antenna through wireless transmission.

6. The system according to claim 3, further comprising:
a wire harness connected to said medical monitor and said processing system.

7. The system according to claim 3, wherein
said medical monitor is housed in the first chassis, and
said processing system, said converter, and said analysis processor are housed in a second chassis.

8. The system according to claim 7, wherein said first chassis is connected to said second chassis through a wire harness.

9. The system according to claim 7, wherein said first chassis includes an interface, and said second chassis includes an interface.

10. The system according to claim 9, wherein said interface of said first chassis and said second chassis is selected from a group consisting of an antenna and an array of jacks.

11. The system to claim 3, wherein said processing system is a signal distribution system.

12. The system according to claim 3, wherein said processing system including a switch at least one means for filtering, conditioning, and amplifying a signal.

13. The system according to claim 3, wherein said processing system includes a switch array for switching inputted signals to a monitor.

14. The system according to claim 3, further comprising a calibrator in communication with said signal distribution system.
